(19) United States
(12) Patent Application Publication
(10) Pub. No.: US 2025/0127965 A1
(43) Pub. Date: Apr. 24, 2025

(54) Tissue Regeneration Particles with a Controlled Structure

(71) Applicant: Biogennix, LLC, Irvine, CA (US)

(72) Inventor: Mark Borden, Boulder, CO (US)

(21) Appl. No.: 18/687,537

(22) PCT Filed: Aug. 31, 2023
(86) PCT No.: PCT/US2023/031686

§ 371 Date: Feb. 28, 2024

Related U.S. Application Data
(60) Provisional application No. 63/402,773, filed on Aug. 31, 2022.

(51) Int. Cl. A61L 27/56; A61L 27/38; A61L 27/54; A61L 27/58; B33Y 80/00

(52) U.S. Cl. CPC A61L 27/56; A61L 27/38; A61L 27/54; A61L 27/58; B33Y 80/00; A61L 2300/406; A61L 2300/41; A61L 2300/414; A61L 2300/64; A61L 2400/06; A61L 2430/02

(57) ABSTRACT

Disclosed herein are tissue regeneration materials and methods of production and use thereof. Particles with a controlled structure that improves the tissue regeneration process by providing more space for tissue ingrowth are disclosed.

### CROSS-REFERENCE TO RELATED APPLICATION

This application claims the benefit of, and priority to, U.S. Provisional Patent Application Ser. No. 63/402,773, filed on Aug. 31, 2022, the entire disclosure of which is hereby incorporated by reference in its entirety.

### FIELD

The present Specification relates to the production and use of tissue regeneration materials.

### BACKGROUND

When certain tissues in the body are damaged, localized healing and tissue regeneration can occur. However, the body's ability to form new tissue in these areas is subject to certain limitations. One main limitation is the size of the area where new tissue formation needs to occur. Tissue regeneration is typically initiated at the periphery of the damaged area or void, with new tissue filling in from these edges. However, if an area is too large, the healing process will eventually stop, resulting in a partial fill of new tissue. To address this issue, tissue regeneration materials (such as bone graft materials) are used to surgically treat an injury or defect in order to help facilitate the body's natural healing process. This is accomplished by providing a physical structure or “scaffold” to support new tissue formation across the entire area.

Tissue regeneration materials are surgically implanted in an area of the body that would not heal sufficiently without intervention. For porous materials, once the material is implanted, new tissue will form on the surface of the scaffold and through the porosity. Non-porous materials can also be used. However, non-porous materials need to be in a small particle form that allows tissue growth around the particles and in the spacing between the particles (i.e. the inter-particle space). The presence of porous or particulate materials in the tissue void provides a support structure that enables tissue formation throughout the entire area and allows the site to fully heal.

In tissue regeneration surgical procedures, surgeons typically prefer materials that have moldable, flexible, and/or injectable properties. These materials are easy to use and have the ability to conform to the irregular volume of an implantation site. Products with these properties are typically created by combining tissue regeneration particles or granules with a moldable, flexible, or injectable carrier that is resorbed shortly after implantation. In these formulations, the particle/granule is the core component, functioning as a tissue regeneration scaffold, while the temporary carrier serves to improve intraoperative handling and placement.

Current tissue regeneration particles are formed through various techniques that create porous or solid particles, but there remain numerous shortcomings associated with these particles. For example:

### Porous Particles/Granules:

a. There are a number of shortcomings related to the production of porous particles or granules for tissue regeneration applications. These are primarily based on variable porosity, structural/mechanical issues, and production yields.
b. Porosity
c. Porosity within a tissue regeneration particle provides an increased surface area for cell attachment and allows more tissue to form within an implant site. However, the techniques used to make these structures commonly result in porosity with a high degree of variability. While certain parameters of pore forming techniques can be controlled, the resulting structure is not precisely created and lacks an overall uniformity. Specifically, current processes typically create structures with a broad range in pore size. This range can limit the healing response, because small pores can prevent tissue ingrowth and large pores can slow down or stop the tissue formation process. Additionally, it is difficult to control interconnectivity between pore channels with existing techniques. This is an important aspect of porosity because it directly relates to how well the scaffold supports tissue formation throughout its entire structure. Pores that end without a connection to a neighbor pore (“dead-end” channels) or pore connections that are smaller than the main pore channel can reduce or limit the tissue ingrowth process.
d. Pore variability is particularly an issue in void forming techniques that use “sacrificial” particles to create pores. In these processes, void forming particles are distributed throughout a target material. The particles are then removed through dissolution, heating, or other methods. The resulting porosity is a negative analog of the volume that the void forming particles previously occupied. In this technique, the shape and size of the individual voids can be controlled, but a uniform and controlled porosity throughout the material cannot be achieved. This is due to the random orientation and distribution of the void forming particles throughout the target material. Further, the creation of a connected pore channel is dependent on the contact points between adjacent void forming particles. Due to this dependency, pore connection points tend to be smaller than the main pore, and there is a higher incidence of “dead-end” channels and isolated pores lacking any interconnectivity.
e. Structural Issues:
f. Although particulate tissue regeneration materials are not load-bearing, they do need to survive manufacturing processes (such as mixing, compounding, syringe filling, etc.), surgeon handling, and implantation. Due to the inverse relationship between porosity and strength, high porosity materials have fragile structures that can easily be crushed into a powder during standard handling. If this occurs, the material can no longer function as scaffold and becomes ineffective. Additionally, standard pore forming techniques can result in variable pore structures with unsuitable properties. This is due to pores that are too small for tissue ingrowth or too large to allow the structure to properly function as a scaffold.
g. 3-D Printed Structures:
h. Three-dimensional (3-D) printing techniques can be used to form porous structures for tissue regeneration. Due to use of a CAD-based design (computer aided design), these processes allow precise control over the general structure of the pore system. Implants are typically formed using a line-by-line printing technique to fabricate a single layer at a time. The process is repeated on subsequent layers until the entire 3-D structure is formed. Implants created with this technology have been in the form of various geometric shapes (cylinders, blocks, cubes, etc.) or custom physiological shapes (e.g. jawbone segment, cranioplasty void, etc.). However, the main limitation to these large, bulk implants is that most tissue regeneration surgical procedures are focused on treating much smaller defects. The large pre-formed shapes are not compatible with most surgical procedures due to the need for significant intraoperative customization. This is required to properly size a large bulk implant to an irregular defect. While some 3-D printed implant can be custom created for a patient's anatomy, this is typically associated with load-bearing defects and is not common in most tissue regeneration surgeries. This is due to the limitations of medical imaging, the incompatibility with other surgical implants, and the time associated with creating patient specific implants.
i. For the large majority of tissue regeneration procedures, surgeons prefer to use easily customizable products that typically have moldable, flexible, or injectable characteristics. These tissue regeneration product forms are typically composed of particulate or granulated materials (˜0.5 to 2.0 mm) mixed with a moldable and resorbable carrier. To date, 3-D printing techniques have not been used to create individual tissue regeneration particles/granules. Previously, this has been limited by 3-D techniques with lower resolution or slow printing times not compatible with mass production of particles.
j. Porous Material Granulation:
k. Porous tissue regeneration materials are typically manufactured in a block form, and then processed into smaller particles/granules. Various methods are used to fragment, cut, or crush the blocks in order to reduce the size and create porous particles or granules. Although the core concept is to break apart the porous block, the structure inevitably gets partially crushed, thereby creating a powder and resulting in unusable material. As a result, these production processes result in low yields and generate a significant amount of waste.

### Solid Particles:

a. Certain tissue regeneration products utilize solid particles as a scaffold for tissue formation. Although there is no porosity within the particle, the packing of the particles in 3-D space creates inter-particle spacing. In tissue regeneration applications, the new tissue growth will occur on the surface of the particles and within this inter-particle space. This allows the packed particles to act as an effective tissue regeneration scaffold. While particle spacing can be roughly controlled by the particle shape and size, overall control of the open space between the particles is limited and only allows for minor modification. For example, a moldable product with particles suspended a carrier would have greater inter-particle spacing, if a low volume of particles is used. However, following implantation, resorption of the carrier would cause the particles to collapse into a tighter packing configuration, and the inter-particle space would be reduced along with the implant volume. This would result in a partial fill of the defect and a limited tissue formation response.
b. Additionally, the shape of solid particles can be problematic for tissue regeneration applications. Solid particles can be created by fracturing, grinding, or crushing larger pieces of materials. This generates an irregular particle shape with a high degree of variability and can create a significant number of particles with flat surfaces. Although sieving techniques can be used to control particle size, this is not exact due to the random nature of having elongated particles pass through uniform sieve openings. Additionally, even with moderate control over particle size, the particles are still highly irregular and variable. In tissue regeneration applications, the packing of irregular particles into a tissue defect, especially those with flat surfaces, can be highly problematic. This is due to the tight spacing between particles that can significantly limit or delay the tissue ingrowth process. Although rounded or spherical particles eliminate this issue, control over the inter-particle space is still limited, as explained above.

As a result of the shortcomings associated with both porous and solid particles, improved materials and methods are desirable.

### SUMMARY

The instant disclosure provides improved particles, compositions, and methods for tissue regeneration.

Disclosed compositions comprise particles, for example particles created using CAD techniques and produced, for example, via high resolution 3-D printing, stereolithography techniques, injection molding, or other techniques, to create individual tissue regeneration particles or granules with a controlled particle shape and porosity. Porosity can be in the form of pores penetrating through the particle and/or structured as grooves or channels on the particle surface. Thus, disclosed particles comprise consistent, specific characteristics enabling better tissue regeneration performance due to increased tissue regeneration space.

Disclosed embodiments describe compositions comprising particles with a controlled structure produced using high resolution 3-D printing, stereolithography techniques, injection molding, or other techniques to create individual particles or granules with a CAD-based particle shape, porosity, and surface features (for example, grooves). Particles can also be further processed to create a surface treatment on the entire particle or in select areas, such as the porosity or surface feature. Surface treatments can be chosen to improve the tissue formation response. Thus, disclosed particles comprise consistent, specific characteristics enabling better enabling better tissue regeneration performance due to an enhanced surface.

Further embodiments comprise particles with specific interlocking and flowability characteristics. For example, design of particle grooves can include intersection points where two or more grooves can meet, as seen in the TOP VIEW in FIG. 5. This intersection point creates a larger area where the particles can interlock. This degree of interlocking can be modulated to increase or decrease the flowability of the particles.

Disclosed embodiments describe compositions comprising particles with a controlled structure produced using high resolution 3-D printing, stereolithography techniques, injection molding, or other techniques to create individual particles with a CAD-based particle shape and porosity that are specifically designed to function as a pharmaceutically-acceptable drug delivery carrier. Thus, disclosed particles comprise consistent, specific characteristics enabling better pharmaceutical release properties.

Further embodiments comprise methods of making disclosed particles, for example through the use of 3-D printing such as DLP (digital light projection) printing or stereolithography.

Further embodiments comprise methods of making disclosed particles, for example through the use of injection molding.

Further embodiments comprise kits comprising disclosed particles and compositions.

Further embodiments comprise methods of use of disclosed particles and compositions.

### SPECIFIC EMBODIMENTS INCLUDE

Embodiment 1: A tissue regeneration particle with a controlled structure comprising a rounded or structured surface comprising surface groove(s) comprising a controlled and uniform shape and size.

Embodiment 2: The particle of embodiment 1, wherein the grooves are independent of one another and do not intersect.

Embodiment 3: The particle of embodiment 1, wherein the grooves connect at an intersection point.

Embodiment 4: The particle of embodiment 3, wherein the intersection point is larger area than the groove.

Embodiment 5: The particle of embodiment 1, wherein the grooves comprise a combination of independent grooves and connected grooves.

Embodiment 6: The particle of embodiment 1, wherein the grooves comprise a square or rectangular cross-section.

Embodiment 7: The particle of embodiment 1, wherein the grooves comprise a semi-circular or semi-elliptical cross-section

Embodiment 8: A tissue regeneration particle with a controlled structure comprising a rounded or structured surface comprising a controlled and uniform porosity penetrating through the particle.

Embodiment 9: The particle of embodiment 8, wherein the pores are independent of one another and do not intersect.

Embodiment 10: The particle of embodiment 8, wherein the pores connect at an intersection point.

Embodiment 11: The particle of embodiment 10, wherein the intersection point is larger area than the pores.

Embodiment 12: The particle of embodiment 8, wherein the pores comprise a combination of independent pores and connected pores.

Embodiment 13: The particle of embodiment 8, wherein the pores comprise a square or rectangular cross-section.

Embodiment 14: The particle of embodiment 8, wherein the pores comprise a circular or elliptical cross-section.

Embodiment 15: A tissue regeneration particle with a controlled structure comprising a rounded or structured surface comprising controlled and uniform grooves and controlled and uniform grooves porosity penetrating through the particle.

Embodiment 16: The particle of embodiment 15, wherein the grooves and pores are independent of one another and do not intersect.

Embodiment 17: The particle of embodiment 15, wherein the grooves and pores connect at an intersection point.

Embodiment 18: The particle of embodiment 17, wherein the connection point is larger in area than the pores.

Embodiment 19: The particle of embodiment 15, wherein the grooves and pores comprise a combination of independent and connected grooves and pores.

Embodiment 20: The particle of embodiment 15, wherein the pores comprise a square or rectangular cross-section.

Embodiment 21: The particle of embodiment 15, wherein the pores comprise a circular or elliptical cross-section.

Embodiment 22: A tissue regeneration particle of embodiments 1, 8, and 15 where the particle is surface treated.

Embodiment 23: A particle of embodiment 22 wherein the treatment covers the entire surface.

Embodiment 24: A particle of embodiment 22 wherein the treatment covers a portion of the surface.

Embodiment 25: A tissue regeneration particle of embodiments 1, 8, or 15 wherein a therapeutic material is added to the surface of the particle.

Embodiment 26: A particle of embodiment 25 wherein the therapeutic material comprises growth factors, antibiotics, anti-inflammatory compounds, or cells.

Embodiment 27: A tissue regeneration particle of embodiments 1, 8, or 15 wherein the particles are created using three-dimensional printing techniques or stereolithography.

Embodiment 28: A tissue regeneration particle of embodiments 1, 8, or 15 wherein the particles are created using injection techniques.

Embodiment 29: A tissue regeneration product wherein the particles of embodiments 1, 8, or 15 are combined with a carrier to create a moldable mixture.

Embodiment 30: A tissue regeneration product wherein the particles of embodiments 1, 8, or 15 are combined with a carrier to create a flexible mixture.

Embodiment 31: A tissue regeneration product wherein the particles of embodiments 1, 8, or 15 are combined with a carrier to create an injectable mixture.

Embodiment 32: The particle of any preceding embodiment, wherein said particle comprises <50 μm resolution in the X, Y, and Z axes.

Embodiment 33: The particle of embodiment 32, wherein said resolution comprises X-Y axes resolution of 35 μm or less.

Embodiment 34: The particle of embodiment 33, wherein said resolution comprises a Z-axis resolution of 25 μm or less.

Embodiment 35: A composition comprising a particle of any of the preceding embodiments and a moldable, flexible, or injectable carrier that is resorbed shortly after implantation.

Embodiment 36: A method of performing tissue regeneration comprising implanting a composition comprising:
a tissue regeneration particle with <50 μm resolution in the X, Y, and Z axes; and
a moldable, flexible, or injectable carrier;
wherein said carrier is resorbed shortly after implantation.

Embodiment 37: The method of embodiment 36, wherein said particle comprises X-Y axes resolution of 35 μm.

Embodiment 38: The method of embodiment 37, wherein said particle comprises a Z-axis resolution of 25 μm or less.

Embodiment 39: The method of any preceding embodiment, wherein said particle is made by 3-D printing.

Embodiment 40: The method of embodiment 39, wherein said 3-D printing comprises DLP printing.

### BRIEF DESCRIPTION OF THE DRAWINGS

FIG. 1 shows an example of tissue ingrowth being improved by adding grooves and pores (right image) to solid sphere particles (left image) to create new space for tissue ingrowth (secondary porosity) not present in the solid particle example (primary porosity only).

FIG. 2 shows a porous sphere structure with 4 cylindrical pores spanning through each geometric face of the sphere (left). The cross-sectional image on the right shows how pore connection points can be expanded into a spherical void. Compared to a solid particle, the pores provide new space for tissue ingrowth.

FIG. 3 shows an example of a grooved sphere with interconnecting horizontal and vertical grooves along the sphere axes that provide new space for tissue in-growth that is not present in solid spheres.

FIG. 4 shows an example of a porous and grooved sphere designed to interlock to neighboring particle. Interconnected grooves were created in the horizontal and vertical axes. The pore structure consists of cylinder pores spanning through each geometric face of the sphere. Multiple intersection points (6 total) on the particle surface provide areas where adjacent particles may interlock. Compared to the solid particles, the pores and grooves provide new space for tissue ingrowth.

FIG. 5 shows how the packing of spherical particles with both grooves and pores that are designed to remain flowable. The groove design consists of only vertical pore with a “wave” pattern to reduce interlocking. A single intersection point is found on the top of the particle. Interconnecting pores are found in 5 out of the 6 particles faces. Compared to the solid particles, the pores and grooves provide new space for tissue ingrowth.

FIG. 6 shows surface treatment options resulting in coating of the entire particle or only the secondary porosity.

FIG. 7 shows a flowable particle design for bone graft applications.

FIG. 8 shows initial trial of Digital Light Processing (DLP) printing of the flowable grooved-porous sphere particles. Images show the initial CAD design, a photograph of the actual DLP printed particle, and a scanning electron micrograph of the actual DLP printed particle.

FIG. 9 shows how groove intersection points can result in a particle interlock.

### DETAILED DESCRIPTION

Disclosed compositions comprise tissue regeneration particles, for example particles produced using high resolution 3-D printing, stereolithography techniques, injection molding, or other techniques to create individual particles with a CAD-controlled particle shape, porosity, and surface features (for example, grooves). Thus, disclosed particles display consistent, specific physical characteristics enabling better tissue regeneration performance. Further, by increasing particle consistency with regard to their physical shape, the practitioner can compare the effectiveness of particles having different sizes, shapes, porosity, etc., and thus determine optimal treatment methods.

For example, in certain applications such as bone grafting, porous particles comprising 300 μm grooves might outperform similar particles comprising 100 μm grooves. Disclosed methods provide the ability to consistently produce particles with specific physical characteristics with a level of precision not possible with earlier production techniques.

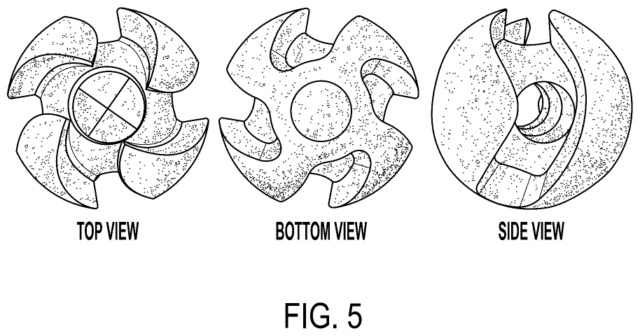

Further embodiments comprise particles designs optimized for 3D printing. The 3-D printing process is a “bottom up” printing process whereby the base of the particle is printed first. In DLP printing, the particle base must attach to the printer's build plate in order to achieve a successful print. Accordingly, the particle base must have sufficient surface area (for example, >100 μm in width) to allow for proper attachment. Thus, disclosed embodiments comprise a “build area” at the bottom of the particles (see BOTTOM VIEW of FIG. 5). This area is printed first and adheres to the printer's build plate. Alternatively, removable support structures can be used to attach the particle to the build plate. Once printed, the particle can be physically or mechanically removed from the support structure and the support structure is discarded.

### Definitions

“Administration,” or “to administer” means the step of giving (i.e. administering) a disclosed composition, material or agent to a subject. The materials disclosed herein can be administered via a number of appropriate routes.

“Controlled structure” means a particle shape (and associated porosity) that is designed using techniques, such as computer-aided-design (CAD), that imparts exact dimensions to the particle structure. This eliminates random or variable structural features.

“Patient” means a human or non-human subject receiving medical or veterinary care.

“Parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

“Pharmaceutically acceptable” or “therapeutically acceptable” refers to a substance which does not interfere with the effectiveness or the biological activity of the active ingredient or therapeutic material and which is not toxic to a patient.

“Pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition into a tissue, or portion of the body. Each carrier must be “acceptable” in the sense of being compatible with the other ingredients of a subject composition, biocompatible (i.e. not injurious to the patient and the localized tissue healing response). In certain embodiments, a pharmaceutically acceptable carrier is non-pyrogenic. Exemplary materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; phospholipids, such as lecithin, and triglycerides; and other non-toxic compatible substances employed in pharmaceutical formulations.

“Pharmaceutical composition” refers to a formulation containing the materials described herein in a form suitable for administration to a subject. In embodiments, the pharmaceutical composition is in bulk or in unit dosage form. The quantity of materials in a unit dose of composition is an effective amount and can be varied according to the particular treatment involved. One skilled in the art will appreciate that it is sometimes necessary to make routine variations to the dosage depending on the age and condition of the patient. The dosage will also depend on the route of administration. In a preferred embodiment, the materials are mixed under sterile conditions with a pharmaceutically acceptable carrier, and with any preservatives, buffers, or propellants that are required. In another preferred embodiment, the materials are mixed with a pharmaceutically acceptable carrier, and with any preservatives, buffers, or propellants that are required, and terminally sterilized using technique such as gamma or electron beam sterilization.

“Porosity” means any feature added to a particle shape that provides additional space for tissue ingrowth. This can be in the form of channels, openings, spaces penetrating the particle or channels, openings, spaces on the surface of the particle (e.g. grooves). One skilled in the art will appreciate that the “porosity” shape, size, and interconnectivity can be tailored to the type of tissue being treated.

“Therapeutically effective amount” means the level, amount, or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured, damaged, or congenitally missing tissue, or by altering, changing, enhancing, improving, ameliorating and/or beautifying an existing or perceived disease, disorder or condition.

The inventor has partially overcome solid particle issues in the bone graft market by improving particle geometry and packing by using spheres (see U.S. Pat. Nos. 8,506,981 and 8,871,235; incorporated by reference herein). However, due to the inherent configuration of solid particle packing, modification to or control over the interparticle space is limited, as such areas for tissue ingrowth continue to remain limited to, for example, less than 50%.

The present disclosure addresses the issues associated with both porous and solid particles used as a tissue regeneration materials. For example, disclosed embodiments comprise the use of high resolution 3-D printing, stereolithography techniques, injection molding, or other techniques to consistently create individual particles with a CAD-based particle shape and porosity. Thus, disclosed particles comprise specific characteristics enabling better tissue regeneration performance.

### Particles

Disclosed herein are particles with a controlled structure comprising surface features. For example, disclosed particles can comprise grooves, slots, channels, indentations, furrows, and combinations thereof. For example, embodiments can comprise particles comprising grooves of various widths, such as 50 μm, 100 μm, 150 μm, 200 μm, 250 μm, 300 μm, 350 μm, 400 μm, 450 μm, 500 μm, or the like.

In embodiments, the grooves of a particle can be of a uniform or non-uniform width. In embodiments comprising uniformly-sized grooves, the grooves of a particle can comprise widths within 1%, 1.5%, 2%, 2.5%, 3%, 3.5%, 4%, 4.5%, or 5% of each other. Grooves can also comprise larger differences in size within 10%, 20%, 30%, 40%, or 50% of each other. The grooves can also vary in the number. Single grooves or multiple grooves are envisioned. In embodiments with multiple grooves, grooves may be independent of one another with no connecting points, or may connect with additional grooves at one or more locations. Connection points can also be designed with specific geometry. In one embodiment, connection points can be larger than the associated grooves and/or have unique shapes (e.g. spherical connection point).

Disclosed herein are particles comprising porosity within the particle. Pore size can vary in disclosed embodiments. For example, in embodiments, the pore diameter can be 50 μm, 100 μm, 150 μm, 200 μm, 250 μm, 300 μm, 350 μm, 400 μm, 450 μm, 500 μm, 550 μm, or the like. In embodiments, the pores of a particle can be of a uniform or non-uniform diameter. In embodiments comprising uniformly-sized pores, the pores of a particle can comprise diameters within 1%, 1.5%, 2%, 2.5%, 3%, 3.5%, 4%, 4.5%, or 5% of each other. Pores can also comprise larger differences in size within 10%, 20%, 30%, 40%, or 50% of each other. In embodiments, pores can be substantially or completely spherical in shape.

The pores can also vary in number. Single pores or multiple pores are envisioned. In embodiments with multiple pores, pores may be independent of one another with no connecting points or may connect with additional pores at one or more locations. Connection points can also be designed with specific geometry. In one embodiment, connection points can be larger than the associated pores and/or have unique shapes (e.g. spherical connection point).

Disclosed embodiments can comprise a primary, CAD-based particle design with a rounded or structured surface that provides optimal, consistent, and predictable interparticle spacing, and the ability to flow or roll over one another. Spherical particles are described herein, but this term also covers ellipsoids, spheroids, or any other flowable rounded shape. For example, spherical particles provide one of the most uniform primary porosity configurations and have the additional advantage of being more flowable than irregular particles. This is advantageous for products that are moldable, flexible, or injectable.

In embodiments, the particle is comprised of multiple “faces”; for example, a spherical particle can be multifaceted.

Embodiments do not cover shapes with flat surfaces that could align and stack, thereby decreasing inter-particle spacing, or tetrapod or similar shapes that interlock and do not have the ability to flow over one another. These shapes can be less effective for moldable, flexible, or injectable formulations.

However, in certain embodiments, a degree of “interlocking” of particles can be advantageous. This is useful in preventing particle migration or movement following implantation. In these embodiments, a grooved-pore structure of the particles was specifically chosen to provide more area for tissue in-growth. The design of the grooves can include intersection points where two or more grooves can meet. This is seen in the TOP VIEW in FIG. 5. This intersection point creates a larger area where the particles can interlock. With the intersection being one of the main particle interlock areas, changing the number of intersection points in a particle can control the degree to which the particles will interlock. It is advantageous to control the interlocking ability of the particles by modifying the design.

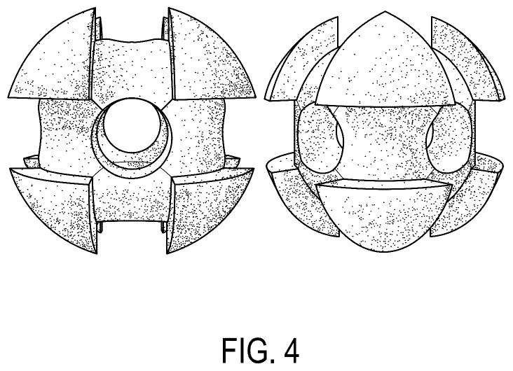

In some applications, minimal interlocking is desirable to create flowable compositions where the particles do not interlock and “roll” over each other. In the design in FIG. 5, particle interlocking was minimized to create a flowable composition. This was achieved by removing the horizontal groove from the design in FIG. 4 and moving the intersection point to the top of the particle. Since this is opposite the build area, it creates only a single intersection point on the particle. Additionally, a “wave” pattern was used in the groove structure instead of a straight channel groove. This minimizes interlocking that could happen along the length of a groove.

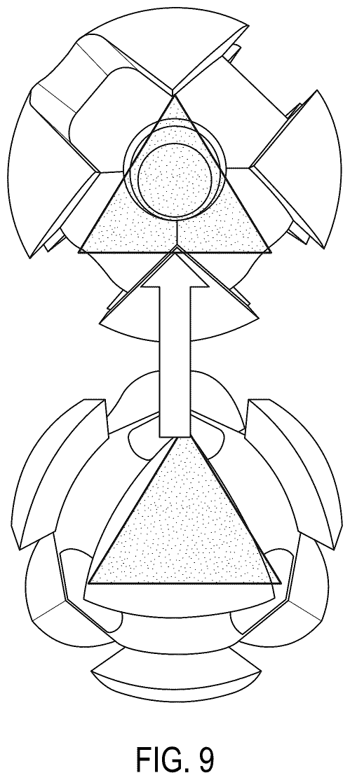

Conversely, embodiments can be designed to increase the ability of the particles to interlock. This can be advantageous applications where particle interlock can provide improved mechanical stability and minimize particle movement and migration. For example, FIG. 4 shows a particle with 6 intersections and straight channel grooves. This increased the ability of the particles to interlock. In further embodiments, the groove placement creates a triangle shaped area that can fit into the intersection resulting in an interlock as seen in FIG. 9. In embodiments, the size of the intersection can be increased to decrease the flowability of the particles, or decreased to increase the flowability of the particles.

The particle shape envisioned by the invention includes designs with rounded surfaces. This allows for improved interparticle spacing and packing. This is advantageous over shapes that have one or more flat surfaces that could pack together with little to no particle spacing.

In embodiments, the shape is not a tetrapod.

In embodiments, the shape is not a pyramid.

In embodiments, the shape is not a cone.

In embodiments, the shape is not a cylinder.

In embodiments, the shape is not a cube with a square or rectangular shape.

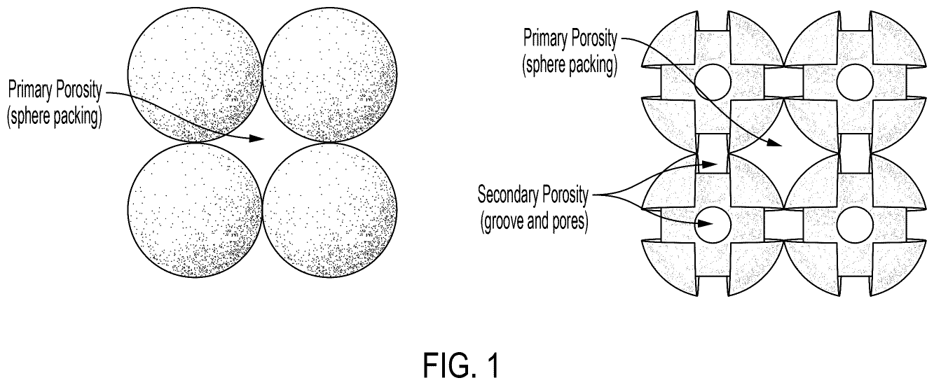

In addition to the primary particle design, embodiments must have a secondary porosity to provide more room for tissue ingrowth. As previously discussed, the packing of particles in a 3-D volume creates spacing between the particles that allows for tissue in-growth. This spacing is called the “primary porosity” with a particulate-based tissue regeneration product. For solid particles, this is the only porosity available for new tissue formation. For porous particles, the inter-particle spacing provides a primary porosity while the pores within the particle provide a “secondary porosity”. The advantage of the secondary porosity is that it provides additional space for tissue formation (as seen in FIG. 1).

In one embodiment, primary and secondary porosity is accomplished by using spherical particles with surface grooves and/or interconnected pores running through the particle.

Disclosed embodiments capitalize on the improved packing associated with the spherical or rounded shape, and adds additional space (secondary porosity) for tissue ingrowth due to the presence of surface grooves and pores.

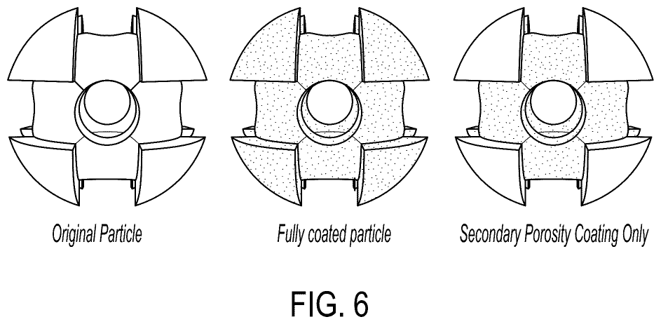

In embodiments, disclosed particles can also contain a surface treatment. The surface treatment can be on the entire particle surface including pores and grooves, or only on select areas of the structures, such as pores and grooves. This is seen in FIG. 6. In these embodiments, the surface treatment can provide enhanced healing for a tissue formation response.

In certain embodiments, disclosed particles can comprise drugs, for example antibiotics, such as in the form of a coating. In these embodiments, the coating can provide a time-release of the coated material. The secondary porosity in these embodiments can be tailored for precise control over the drug release profile. In certain embodiments, the secondary porosity is only used for drug release and not intended for tissue ingrowth.

### Methods of Particle Production

Disclosed methods of particle production comprise the use of Computer-Aided Design or CAD to create particles with a specifically designed and controlled structure. Embodiments comprising the use of CAD enable the practitioner to precisely control the particle shape and size. Additionally, the use of CAD allows for precise control over the shape, size, and interconnectivity of the surface features and pores. The direct control of the particle design through CAD thereby eliminates the variability inherent to current techniques.

By imparting this type of structural control, particles can be specifically engineered to provide an improved tissue regeneration response that is capable of forming tissue in a greater amount and/or at a faster rate. This is due to the creation of a secondary porosity when particles with grooves and/or pores are packed together. Compared to solid particles, the secondary porosity provides more space for tissue in-growth. Structural control over the particles also provides the ability to modify or enhance material properties such as dissolution, material ion release, resorption, and/or strength. Further, structural control allows for the creation of advanced particles with certain surface features intended to improve or accelerate the tissue formation response and/or impart advantageous properties.

Disclosed methods of particle production can comprise 3-D printing. The use of 3-D printing to create tissue regeneration scaffolds has been previously limited to larger geometric or physiologic shapes with lower part resolution. Additionally, fabrication output has been relatively slow, making it unacceptable for the production of significant part quantities for tissue regeneration products. Earlier 3-D printing technology, such as PolyJet or MultiJet printing, fused deposition modeling, selective laser sintering, electron beam melting, has utilized line-by-line processes to create parts with a relatively slow fabrication rate. Further, certain printer technology was not able to produce micron-sized features with an acceptable surface resolution. To date, the combination of slow printing speeds and low resolution has made the printing of individual tissue regeneration particles unachievable due to unacceptable surface finish, or prohibitively time consuming and expensive manufacturing processes. This has made these techniques incapable of producing production-level quantities of individual particles (such as 1M particles/year) needed for tissue regeneration products. In contrast, disclosed embodiments comprise rapid production of tissue regeneration materials using high-resolution, CAD-based fabrication techniques.

For example, in embodiments comprising 3-D printing and stereolithography techniques, part resolutions can comprise less than, for example, 200 μm, 150 μm, 100 μm, 50 μm, or 25 μm.

In particular, disclosed embodiments employing 3-D printing using DLP (digital light projection) can achieve <50 μm resolution in both the X-Y plane (cross-sectional resolution) and Z-axis (layer height). This advancement in 3-D printing technology can now produce particles with a smooth surface and micron-sized surface features and porosity. An additional advantage of DLP printing is that the part design is built using an entire cross section of the part, rather than line-by-line method. In this process, multiple individual particles can be fabricated in DLP printer build area at the same time to maximize production output. Once of the key advantages to DLP printing is that the entire build area cross-section is projected at once. This builds all parts on the build platform layer by layer at the same time. As a result, this method results in a significant increase in part production due to a significantly decreased in printing time, compared to line-by-line methods.

In 3-D printing DLP and stereolithography embodiments, disclosed particles are made from a photopolymerizable resin. This resin comprises biocompatible and biodegradable polymers and copolymers that are photopolymerizable. In other embodiments, the resin is loaded with ceramic or bioactive glass. In these embodiments, the resin is a temporary material that is fully removed during additional processing and is not required to be biocompatible or biodegradable. During 3-D printing with a ceramic or glass loaded resin, the parts are printed, and the resin is removed using a heat treatment or other methods. Resulting ceramic or glass particles are then sintered to fully fuse the material and create a structurally sound part.

In embodiments, disclosed particles with a controlled structure can comprise bioceramics such as Akermanite, tricalcium phosphate, hydroxyapatite, or biphasic calcium phosphate, and/or bioactive glasses such as 1393 bioactive glass or 45S5 bioactive glass. Various combinations of these materials are also envisioned. For example, disclosed embodiments can comprise a combination of, for example, a ceramic/glass and a photopolymerizable resin.

Formulation of the ceramic resin is a critical part of DLP printing. This is mainly based on the ceramic particle size. This is based on having particles smaller than the layer height. Otherwise, layer with an uneven height will be formed which results in poor particle resolution For example, if printing a 25 μm layer, the ceramic particles must be <25 μm and preferably <20 μm (80% or less than the original size), or more preferably <15 μm (60% or less than the original size). The smaller particles also help with maintaining a homogeneous suspension in the resin during printing.

In other 3-D DLP printing and stereolithography embodiments, a biocompatible based photopolymer is used. In these embodiments, such polymers can comprise biodegradable polymers, such as polyhydroxy acids [poly lactic acid (PLA) and/or polyglycolic acid (PLG)], poly(ε-caprolactone) (PCL), poly(trimethylene carbonate) (PTMC), poly(propylene) fumarate (PPF) in combination with diethyl fumarate (DEF), and combinations thereof. Additionally, bioactive glass or bioceramics may be added to the biodegradable polymer to create composite particles.

The disclosed embodiments also envision other CAD-based micro-fabrication methods capable of mass-producing individual particles with a controlled structure. In an additional embodiment, micro-injection molding methods can be employed to make particles with a controlled structure. This is specifically suited for ceramic or glass particles which can be injected molded using a ceramic or glass loaded resin. In this process, the ceramic/glass loaded resin is injection molded in a cavity that produces multiple controlled structure particles. The particles are then detached from the injection molding sprue/runners, and then heat treated to fully remove the resin and sinter the ceramic/glass. Residual sprue pieces can then be recycled and run through the injection molding process repeatedly.

### Compositions

Disclosed herein are compositions comprising particles with a controlled structure as disclosed herein. Disclosed compositions comprise disclosed particles mixed with a pharmaceutically acceptable carrier. Disclosed compositions comprise moldable or flexible formulations that can be custom shaped to fit the implant site. Disclosed compositions also comprise flowable or injectable formulations, for example flowable compositions suitable for administration through a cannula, such as via extrusion, or injectable composition suitable for administration through a needle, such as via percutaneous injection.

### Commercial Products/Kits

The present tissue regeneration material can be finished as a commercial product by the usual steps performed in the present field, for example by appropriate sterilization and packaging steps. For example, the present material may be packaged in syringes, cannulas, or containers that are sealed in pouches or trays, and then terminally sterilized by gamma or beta irradiation.

Disclosed kits, such as for use in surgery and/or in the treatment of injuries and/or wounds, can comprise a disclosed tissue regeneration material and at least one administration device, for example syringe, cannula, and/or minimally invasive delivery device.

In certain embodiments, a pharmaceutical compound can be included for absorption on the particles or in a moldable, flexible, or injectable product. This comprises anti-bacterial agents, immunosuppressive agents, anti-inflammatory agents, anti-fibrinolytic agents, especially aprotinin or ECEA, growth factors, vitamins, cells, or mixtures thereof.

The kits are designed in various forms based on the specific deficiencies they are designed to treat.

### EXAMPLES

The following non-limiting Examples are provided for illustrative purposes only in order to facilitate a more complete understanding of representative embodiments. This example should not be construed to limit any of the embodiments described in the present specification. In the following descriptions, bone graft particles are described for such illustrative purposes. However, these concepts can be applied to any tissue regeneration particle.

### Example 1

### Production of Tissue Regeneration Particles

Based on the concept of a CAD-based controlled particle with a spherical shape and secondary porosity, the following embodiments are envisioned:

Embodiment #1 (Porous Particles): Utilizing the spherical particle design, a secondary porosity is created by forming pore channels completely through the particle.

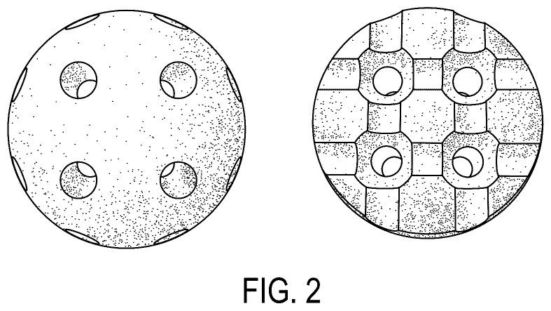

These channels can be independent of one another or, preferably, are interconnected. Interconnected pores can also be designed with a larger connection space to facilitate tissue ingrowth and capitalize on material properties, such as dissolution-based ion release. The pore channels can also be any shape in cross-section. Preferably, the pore channels are sized to provide an optimal tissue ingrowth response. An example is shown in FIG. 2. In this design, four fully penetrating pores are positioned on each face of the sphere. The pores are designed to be connected with the connection point in the shape of a spherical void that is larger than each pore.

### Example 2

### Production of Tissue Regeneration Particles

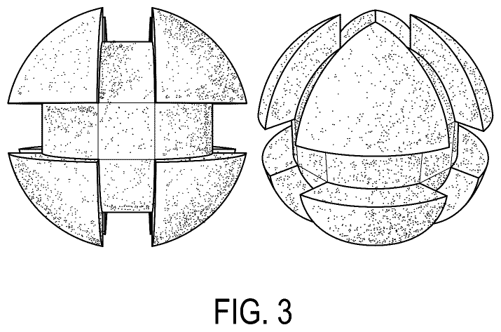

Embodiment #2 (Grooved Particles): In this embodiment, the secondary porosity within the spherical particle is achieved by designing tissue ingrowth grooves into the surface of the particle. These grooves can be independent of one another or, preferably, interconnected. Groove channels can be any shape in cross-section. Preferably, the grooves are sized to provide an optimal tissue ingrowth response. An example is shown in FIG. 3. In this design, a single horizontal and vertical groove is placed on the central axis. The grooves connect on each face of the sphere.

### Example 3

### Production of Tissue Regeneration Particles

Embodiment #3 (Interlocking Grooved and Porous Particles): Using a spherical particle, this design employs both grooves and connecting pores to create a secondary porosity. The advantage of a structure with both grooves and pores is that the secondary porosity can be further increased in volume without excessively increasing the size of the individual groove or pore features. Additionally, the use of multiple groove intersection points creates particles that have a propensity to interlock. An example is shown in FIG. 4. In this design, a single horizontal and vertical groove is placed on the central axis. The grooves connect on each face of the sphere. At the connection pointes, a circular pore is placed through the particle. The pores connect in the center of the particle which creates a hollow spherical space that is larger than the pores. The advantage of this design is that it creates two sources of secondary porosity (see FIG. 1). One space is created from the grooves on the surface, while the other space is created from the interconnected pores. The combination of these spaces provides a significant increase in the volume available for tissue in-growth, compared to a solid particle.

### Example 4

### Production of Tissue Regeneration Particles

Embodiment #4 (Flowable Grooved and Porous Particles): Using a spherical particle, this design employs both grooves and connecting pores to create a secondary porosity. However, a vertical groove design with a “wave” pattern is utilized to minimize particle interlocking and maintain flowability. An example is shown in FIG. 5. This particle was specifically designed to reduce particle interlocking in order to create a flowable tissue regeneration composition. This was achieved by removing the horizontal groove and only using vertical grooves. This creates a single intersection point at the top of the particle compared to 6 intersection points found in the interlocking design shown in FIG. 4. Additionally, a “wave” pattern was used in the groove structure instead of a straight channel groove. This minimizes interlocking that could happen along the length of a groove. Additional tissue ingrowth space was also created by placing pores on 5 out of 6 particle faces with a central intersection point that had a spherical shape larger than the pores. Similar to Embodiment #3, this design also increases the volume available for tissue ingrowth with two sources of secondary porosity.

In Embodiments 1, 2, 3, and 4, grooves and/or pore channels are used to create the secondary porosity. It is known in the art that the shape and size of the porosity can impact the tissue in-growth response into these areas. In general, pores that are too small can prevent tissue ingrowth while pores that are too big can slow down the tissue formation response. Based on the above concepts, the pore shape and size can be controlled and optimized to elicit the best tissue healing response.

### Example 5

### Production of Tissue Regeneration Particles

Embodiment #5 (surface treatment): In this embodiment, particles with a controlled structure are further processed to create a surface treatment on the porosity surface. The surface treatment can be a bioactive coating, nanocrystalline coating, drug delivery coating, or other coatings intended to improve the tissue formation response or impart advantageous properties (e.g. antibiotic release).

Embodiment 5A: In one example in the bone grafting field, a nanocrystalline surface is created on the particle surface. Materials with these types of surface features have been shown to stimulate and accelerate cellular bone formation. This can be created on bioactive glasses (such as Bioglass) or bioactive ceramics (such as Akermanite). Using a controlled particle structure composed of a bioactive material, the nanocrystalline surface is created by soaking the particles in a solution called simulated body fluid. This solution contains solubilized ions that mimic the ions found in body fluid. Once immersed, a nanocrystalline hydroxycarbanoapatite layer forms on the entire exposed surface. In this example, these coated particles can then be used in a bone graft product. For moldable products where the particles are combined with a moldable, resorbable carrier, mixing and handling may remove the layer on the outer particle surface. However, since the grooves and/or pores are protected from rubbing on adjacent particles, these areas will remain coated (FIG. 6).

Embodiment 5B: While the structure of the secondary porosity can be designed primarily for tissue ingrowth, the secondary porosity can also be designed to control specific properties, such as drug release. In this embodiment, drug release becomes the primary design criteria for the secondary porosity rather than tissue ingrowth. The groove/pore channels in drug delivery particles can be sized to directly affect the release rate and release profile of a drug-based coating, in order to maintain an effective therapeutic dosage. Drug coatings can be achieved by standard methods in the art including binding, drying, and adsorption.

### Example 6

### Production of Tissue Regeneration Particles

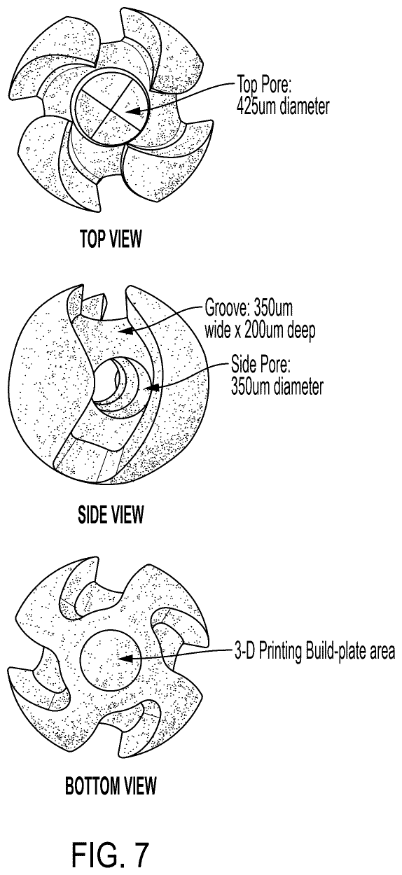

A ceramic particle with a controlled structure was designed for bone grafting applications using the flowable design in Example 4. The particle had an overall spherical shape with a 1.25 mm diameter and is shown in FIG. 7. As described above (embodiment 4), vertical grooves were created in the sphere surface. Grooves were 350 μm wide×200 μm deep and had a “wave” pattern design. These dimensions represent openings that are in the optimal range for bone in-growth (150-500 μm). Additionally, a 350 μm wide pore was created along the X, and Y-axes. The single pore at the groove intersection point on the top of the particle was made larger at 425 μm. A “build-plate” feature was also added to the bottom of the particle to facilitate attachment to the 3-D DLP printer build platform. Compared to a solid sphere, the grooves and pore channels in this design added 56% porosity to the sphere. This represents a significant tissue ingrowth improvement over a solid sphere that has 0% porosity.

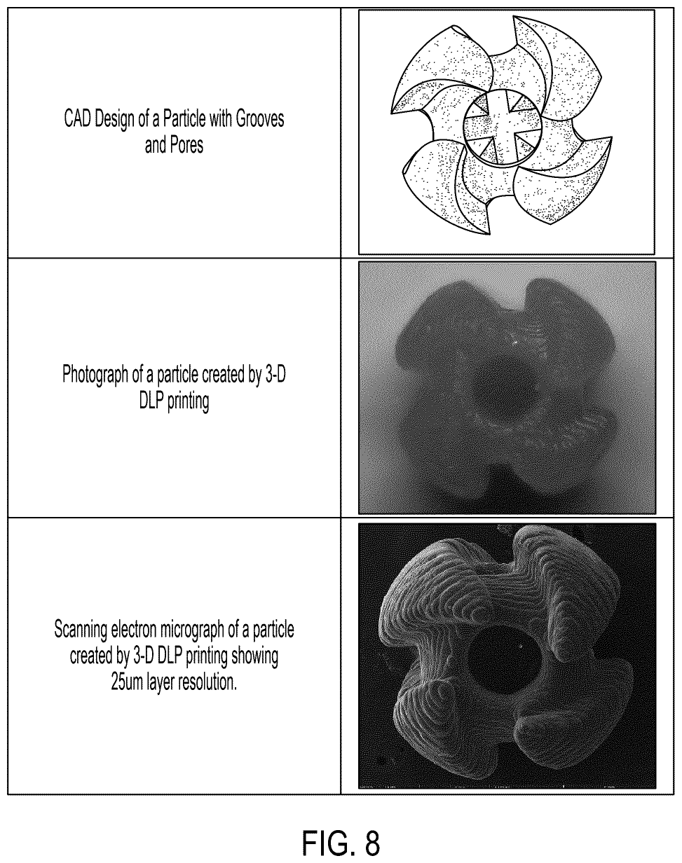

Using this design, particles were printed on a high resolution DLP printer with an X-Y resolution of 35 μm and a 25 μm Z-axis layer. A photopolymerizable resin containing hydroxyapatite (a common bone graft ceramic) was used to create the “green” parts. Green parts were then heat treated in a furnace to burn off the resin and sinter the ceramic. Images of the printed parts are shown in FIG. 8. The trial showed that the printer was capable of creating the CAD-based structure and achieving an acceptable surface resolution. Grooves and pores were clearly seen in the structure.

### Example 7

### Preparation of a Moldable Tissue Regeneration Composition

Particles disclosed herein are mixed with a moldable carrier that is resorbed shortly after implantation. In this example, the particles are combined with a viscous carrier. After addition of the particles, the resulting material has a doughy, moldable consistency that is suitable for easy placement and provides good intra-operative handling. In this type of product, a formulation of 50-90% particles and 10-50% carrier can be used to maintain effective particle filling volume once implanted. Once mixed, the moldable material is packaged and sterilized. During surgery, the moldable material can be directly placed at the implant site and conforms to the irregular defect shape.

### Example 8

### Preparation of a Flexible Tissue Regeneration Composition

Particles disclosed herein are mixed with a flexible carrier that is resorbed shortly after implantation. In one example in the bone grafting field, particles can be combined with a water-based dispersion of collagen fibers. The resulting slurry is then cast into a mold and freeze-dried to remove the water. In this type of product, a formulation of 50-98% particles and 2-50% collagen carrier can be used to maintain effective particle filling volume once implanted. The freeze-dried form can then be packaged and sterilized. During surgery, the freeze-dried form is immersed in liquid to hydrate the collagen and create a flexible tissue regeneration implant. The flexible material can be directly placed at the implant site and conforms to the irregular defect shape.

### Example 9

### Preparation of an Injectable Tissue Regeneration Composition

Particles disclosed herein are mixed with an injectable carrier that is resorbed shortly after implantation. In this example, the particles are combined with a low viscosity, flowable carrier. After addition of the particles, the resulting material has a gel-like, fluid consistency that is suitable for injection. In this type of product, a formulation of 30-90% particles and 10-70% carrier can be used to maintain effective particle filling volume once implanted. The injectable material is then packaged in a syringe or cannula. During surgery, the injectable material can be directly placed at the implant site and conforms to the irregular defect shape.

### Example 10

### Use of Tissue Regeneration Compositions

A disclosed tissue regeneration composition is administered to a physiological location in need of tissue regeneration.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the disclosed embodiments.

Unless otherwise indicated, all numbers expressing a characteristic, item, quantity, parameter, property, term, and so forth used in the present specification are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and embodiments are approximations that may vary. At the very least, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation.

What is claimed is:

1: A tissue regeneration particle with a controlled structure comprising a rounded or structured surface comprising surface groove(s) comprising a controlled and uniform shape and size.

2: The particle of claim 1, wherein the grooves are independent of one another and do not intersect.

3: The particle of claim 1, wherein the grooves connect at an intersection point.

4: The particle of claim 3, wherein the intersection point is larger area than the groove.

5: The particle of claim 1, wherein the grooves comprise a combination of independent grooves and connected grooves.

6: The particle of claim 1, wherein the grooves comprise a square or rectangular cross-section.

7: The particle of claim 1, wherein the grooves comprise a semi-circular or elliptical cross-section.

8: A tissue regeneration particle with a controlled structure comprising a rounded or structured surface comprising a controlled and uniform porosity penetrating through the particle.

9: The particle of claim 8, wherein the pores are independent of one another and do not intersect.

10: The particle of claim 8, wherein the pores connect at an intersection point.

11: The particle of claim 10, wherein the intersection point is larger area than the pores.

12: The particle of claim 8, wherein the pores comprise a combination of independent pores and connected pores.

13: The particle of claim 8, wherein the pores comprise a square or rectangular cross-section.

14: The particle of claim 8, wherein the pores comprise a circular or elliptical cross-section

15: A tissue regeneration particle with a controlled structure comprising a rounded or structured surface comprising controlled and uniform grooves and controlled and uniform porosity penetrating through the particle.

16: The particle of claim 15, wherein the grooves and pores are independent of one another and do not intersect.

17: The particle of claim 15, wherein the grooves and pores connect at an intersection point.

18: The particle of claim 17, wherein the connection point is larger in area than the pores.

19: The particle of claim 15, wherein the grooves and pores comprise a combination of independent and connected grooves and pores.

20: The particle of claim 15, wherein the pores comprise a square or rectangular cross-section.

21: The particle of claim 15, wherein the pores comprise a circular or elliptical cross-section.

22: A tissue regeneration particle of claims 1, 8, and 15 where the particle is surface treated.

23: A particle of claim 22 wherein the treatment covers the entire surface.

24: A particle of claim 22 wherein the treatment covers a portion of the surface.

25: A tissue regeneration particle of claim 1, 8, or 15 wherein a therapeutic material is added to the surface of the particle.

26: The particle of claim 25 wherein the therapeutic material comprises growth factors, antibiotics, anti-inflammatory compounds, or cells.

27: The tissue regeneration particle of claim 1, 8, or 15 wherein the particles are created using three-dimensional printing techniques or stereolithography.

28: The tissue regeneration particle of claim 1, 8, or 15 wherein the particles are created using injection molding techniques.

29: A tissue regeneration product wherein the particles of claim 1, 8, or 15 are combined with a carrier to create a moldable mixture.

30: A tissue regeneration product wherein the particles of claim 1, 8, or 15 are combined with a carrier to create a flexible mixture.

31: A tissue regeneration product wherein the particles of claim 1, 8, or 15 are combined with a carrier to create an injectable mixture.

32: The particle of any preceding claim, wherein said particle comprises <50 μm resolution in the X, Y, and Z axes.

33: The particle of claim 32, wherein said resolution comprises X-Y axes resolution of 35 μm or less.

34: The particle of claim 33, wherein said resolution comprises a Z-axis resolution of 25 μm or less.

35: A composition comprising a particle of any of the preceding claims and a moldable, flexible, or injectable carrier that is resorbed shortly after implantation.

36: A method of performing tissue regeneration comprising implanting a composition comprising:
a tissue regeneration particle with <50 μm resolution in the X, Y, and Z axes; and
a moldable, flexible, or injectable carrier;
wherein said carrier is resorbed shortly after implantation.

37: The method of claim 36, wherein said particle comprises X-Y axes resolution of 35 μm.

38: The method of claim 37, wherein said particle comprises a Z-axis resolution of 25 μm or less.

39: The method of any preceding claim, wherein said particle is made by 3-D printing.

40: The method of claim 39, wherein said 3-D printing comprises DLP printing.
